(19) United States
(12) Patent Application Publication
(10) Pub. No.: US 2015/0192596 A1
(43) Pub. Date: Jul. 9, 2015

(54) SYSTEMS AND METHODS FOR DETECTION OF ELECTRIC FIELDS, ION EXCHANGE, AND PH USING SPECTRAL SHIFT IN DIAMOND COLOR CENTERS

(71) Applicant: THE TRUSTEES OF COLUMBIA UNIVERSITY IN THE CITY OF NEW YORK, New York, NY (US)

(72) Inventors: Dirk R. Englund, New York, NY (US); Ophir Gaathon, New York, NY (US)

(73) Assignee: THE TRUSTEES OF COLUMBIA UNIVERSITY IN THE CITY OF NEW YORK, New York, NY (US)

(21) Appl. No.: 14/564,445

(22) Filed: Dec. 9, 2014

Related U.S. Application Data
(63) Continuation of application No. PCT/US2013/045631, filed on Jun. 13, 2013.
(60) Provisional application No. 61/659,772, filed on Jun. 14, 2012.

(51) Int. Cl. G01N 33/84; G01N 33/58

(52) U.S. Cl. CPC G01N 33/84; G01N 33/582

(57) ABSTRACT

Techniques for detection of electric and magnetic fields, ion exchange, and pH using spectral shift in diamond color centers are disclosed. In one aspect of the disclosed subject matter, a method to detect a change of an electric field or electrochemical parameter in a solution can include introducing at least one diamond structure, including a color center below a surface of thereof, into the solution.

### CROSS-REFERENCE TO RELATED APPLICATION

This application is a continuation of International Application No. PCT/US2013/045631 filed on Jun. 13, 2013 which claims priority from U.S. Provisional Application Ser. No. 61/659,772, filed Jun. 14, 2012, the contents of each of which are hereby incorporated by reference in their entireties.

### STATEMENT REGARDING FEDERALLY SPONSORED RESEARCH

This invention was made with government support under Grant No. FA9550-12-1-0045 awarded by the Air Force Office of Scientific Research, PECASE. The government has certain rights in the invention.

### BACKGROUND

The disclosed subject matter relates to techniques for detection of electric fields, ion exchange, and pH using spectral shift in diamond color centers.

Certain methods for electric field detection using diamonds are based on perturbation of electron spin properties of a color center that resides inside a diamond crystal. However, complex measurement schemes can require long spin coherence time (˜100 μs) and present a challenge given the crystal quality of commercially available diamond nanocrystals.

Accordingly, there exists a need for an improved technique for detection of electric field or other electrical or electrochemical properties.

### SUMMARY

Systems and methods for detection of electric and magnetic fields, ion exchange, and pH using spectral shift in diamond color centers are disclosed herein.

In one aspect of the disclosed subject matter, methods to detect a change of an electrochemical parameter in a solution are provided. An exemplary method can include introducing at least one diamond structure, including a color center below a surface of thereof, into the solution. An electromagnetic pump field can be applied to the at least one diamond structure. A radiative state of the color center can be monitored by measuring a spectral shift of an emission of photons from the color center. The change of the electrochemical parameter of the solution can be detected based on a predetermined correlation between the spectral shift and the electrochemical parameter. In some embodiments, at least one of a surface charge density or an electron affinity of the at least one diamond structure can be modified.

In some embodiments, the diamond structure can be one of a nanodiamond or a bulk diamond crystal. In some embodiments, the solution can be a biological solution or an ionic solution. In some embodiments, the electrochemical parameter can be one of an electric field, an ionic concentration, or a pH level.

In some embodiments, the color center can be a nitrogen vacancy (NV) center. The method can include monitoring the charge state of the NV center, which can be +2, +1, 0, −1, and −2 electron charges. These charge states can be associated with different emission spectra. For example, the NV center can be in either neutral or −1 electron charge states, which can have distinct emission spectra. In some such embodiments, the method can also include modifying at least one of a surface charge density or an ion affinity of the at least one diamond structure to control the fluorescence spectrum or blinking rate of the NV center to enhance detection of the electrochemical parameter. In other embodiments, the color center can be one of a silicon vacancy or a chromium center.

In another aspect of the disclosed subject matter, systems for detecting a change of an electrochemical parameter in a solution are provided. An exemplary system can include a receptacle, an electromagnetic pump field source, and a monitoring device. The receptacle can be adapted to receive the solution and at least one diamond structure having a color center below a surface of thereof, such that the diamond structure is at least partially submerged in the solution.

In some embodiments, the electromagnetic pump field source can be adapted to apply an electromagnetic pump field to the at least one diamond structure. The monitoring device can be coupled to the receptacle and adapted to monitor a radiative state of the color center by measuring a spectral shift of an emission of photons from the color center to thereby detect the change of the electrochemical parameter of the solution based on a predetermined correlation between the spectral shift and the electrochemical parameter. In some embodiments, a surface charge density and/or an ion affinity of the diamond structure can be modified to enhance detection of the electrochemical parameter.

In another aspect of the disclosed subject matter, methods of fabricating a diamond structure for detecting a change of an electrochemical parameter at a surface thereof are provided and can include providing the diamond structure. At least one color center can be induced below a surface of the diamond structure. At least one of a surface charge density or an ion affinity of the diamond structure can be modified to control a radiative state of the color center to thereby enhance detection of the electrochemical parameter based on a predetermined correlation between a spectral shift of an emission of photons from the color center and the electrochemical parameter. In some embodiments, the color center can be induced by one of nano-implanting or electron radiating.

In some embodiments, the diamond structure can be a nanodiamond. The nanodiamond can be injected into one of a biological cell or a biological tissue, bonded to a tip of a micro-manipulated probe, bonded to a surface of a sample holder, bonded to a wall of a flow cell, or bonded to a tip or a side of an optical fiber. In some embodiments the diamond structure can be a bulk diamond crystal. The bulk diamond crystal can be positioned below a sample or attached to a probe.

The accompanying drawings, which are incorporated and constitute part of this disclosure, illustrate and serve to explain the principles of the disclosed subject matter.

### BRIEF DESCRIPTION OF THE DRAWINGS

FIG. 1A-B are diagrams showing FIG. 1A a neutrally charged radiative state of a nitrogen-vacancy (NV) center in nanodiamond and FIG. 1B a negatively charged radiative state of an NV center in a nanodiamond, in accordance with exemplary embodiments of the disclosed subject matter.

FIG. 2 is a diagram showing the fluorescence spectrum of a neutrally charged radiative state and a negatively charged radiative state of an NV center in a diamond structure.

FIG. 3A-B are diagrams showing FIG. 3A a neutrally charged radiative state of an NV center in a bulk diamond crystal and FIG. 3B a negatively charged radiative state of an NV center in a bulk diamond crystal.

FIG. 4 is a diagram illustrating a method to detect a change of an electrochemical parameter in a solution, in accordance with exemplary embodiments of the disclosed subject matter.

FIG. 5 is a diagram illustrating a method of fabricating a diamond structure for detecting a change of an electrochemical parameter at a surface thereof, in accordance with exemplary embodiments of the disclosed subject matter.

FIG. 6 is a diagram illustrating a system for detecting a change of an electrochemical parameter in a solution, in accordance with exemplary embodiments of the disclosed subject matter.

FIG. 7 is a diagram showing a nitrogen-vacancy (NV) center in diamond, in accordance with exemplary embodiments of the disclosed subject matter.

Throughout the figures, similar reference numerals and characters, unless otherwise stated, are used to denote like features, elements, components or portions of the illustrated embodiments. Moreover, while the disclosed subject matter will now be described in detail with reference to the figures, it is done so in connection with the illustrative embodiments.

### DETAILED DESCRIPTION

Techniques for detection of electric fields, ion exchange, and pH using spectral shift in diamond color centers are presented. Changes in electric fields across a diamond structure can alter the charge state of a color center in the diamond. Similarly, changes in the ionic concentration or pH of a solution or of air can alter the charge state of the color center in the diamond. A change in the charge state of the color center can lead to a shift in the fluorescence spectrum of the color center. The mechanism for the spectral shift can occur because of an induced change in the charge state of the color center, a change in the blinking rate of the color center, or a spectral shift via the Stark shift. The shift in the fluorescence spectrum can be measured to monitor the charge state of the color center. Changes in the electric field or changes in the ionic concentration or pH of a solution can be calculated based on a predetermined correlation between the shift in the fluorescence spectrum and the parameter of interest.

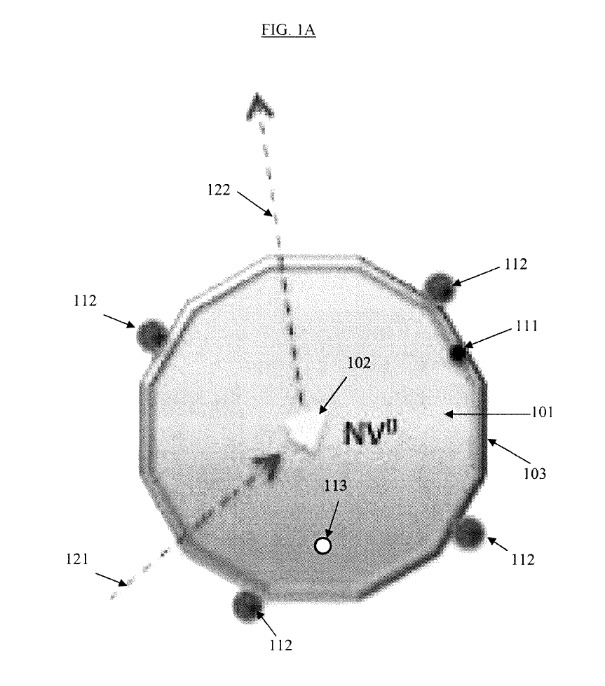

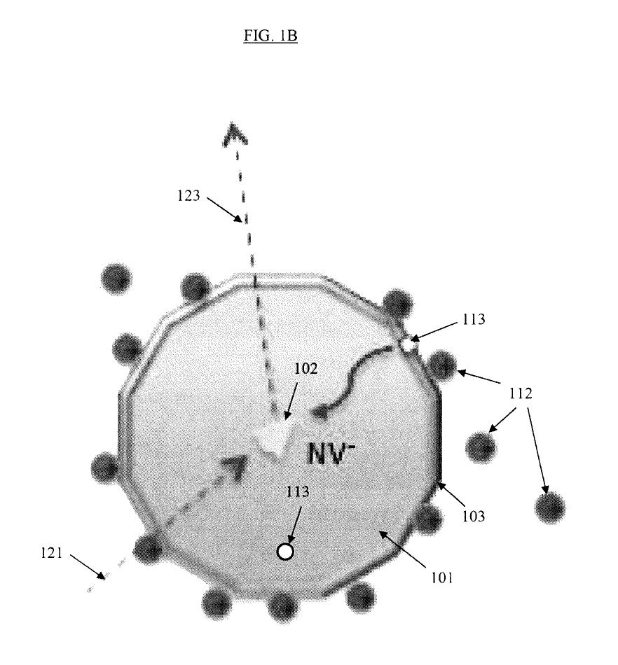

Referring to FIG. 1A, a nanodiamond 101 can have a color center 102. The color center 102 could be any suitable color center, including any atomic defect in a bandgap material. For example, the color center 102 could be a nitrogen vacancy (NV) center, a silicon vacancy, or a chromium center. The color center 102 can be below the surface 103 of the nanodiamond 101. The color center 102 can be any suitable depth below the surface 103 so long as the electric field resulting from the parameter of interest can reach the color center 102, as discussed below. For example, the color center 102 can be between 2 and 30 nm below the surface 103. For example, the color center 102 can be 5-15 nm below the surface 103.

By way of example and not limitation, the color center 102 can be an NV center. Diamond NV color centers can be formed when a substitutional nitrogen and vacancy are created in the carbon lattice, replacing two carbons. Diamond NV centers can occur naturally or can be implanted in a diamond structure via ion radiation or the like. NV center 102 can exist in multiple charge states, including a positively charged radiative state (NV⁺), a neutrally charged radiative state (NV⁰), and a negatively charged radiative state (NV⁻). The charge state of NV center 102 can depend on the electrochemical potential seen by the electrons 111 on the diamond's surface 103 and surrounding environment. For example, the NV⁻ and NV⁰ states can occur when the pH of the surrounding environment is near 7 and the NV center is 5-15 nm below the surface. The NV center can be deeper below the surface, in which case a larger pH can be required to change the NV charge state, thus making the NV charge state selective to pH further from 7.

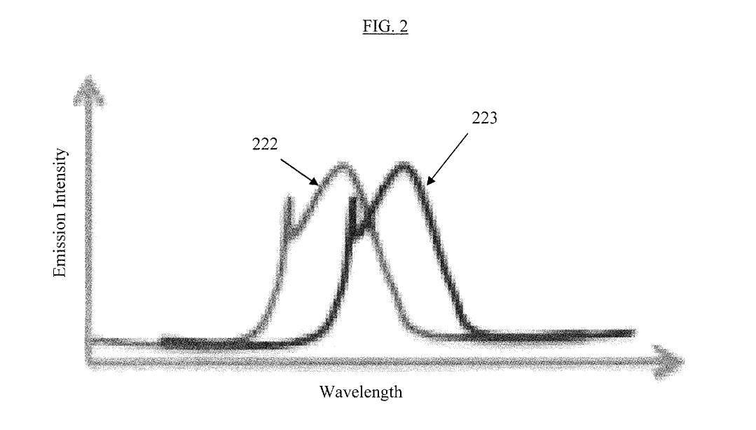

An electromagnetic pump field 121, e.g., light from a laser or light emitting diode, can be applied to color center 102, which in turn can cause color center 102 to emit photons 122. Referring also to FIG. 2, this emission of photons 122 can be detected as the fluorescence spectrum 222. The fluorescence spectrum 222 can depend on the charge state of the color center 102. For example, in connection with an NV center 102, the NV⁻ and NV⁰ states can be relatively bright and efficient light emitters. A monitoring device, for example a photodetector, can be used to optically detect the fluorescence spectrum of NV center 102. A pump electromagnetic field can be applied to the NV center 102. The pump field can be any suitable wavelength. For example, the pump field could have a wavelength of 488-592 nm. As an additional example, the pump field could have a wavelength on the order of 1 μm, utilizing a two-photon absorption process. In another example, a combination of pump wavelengths can be used. Additionally or alternatively, for an NV center 102, the addition of blue photons can elevate an electron 111 into a higher energy state from which it can more easily tunnel into another electron trap 113. Thus, a blue pump can act to liberate the electron on the NV to make the NV more sensitive to electric field changes. For example, when a pump electromagnetic field of 532 nm is applied to NV center 102 in the NV⁰ state, the fluorescence spectrum 122 can have a wavelength peaked near 575 nm and extending to the low 600 nm range. As discussed below with reference to FIG. 1B, when the NV center 102 is in the NV⁻ state, the fluorescence spectrum 223 can have a wavelength peaked over 637 nm and extending to beyond 720 nm. The difference between NV⁰ fluorescence spectrum 222 and the NV⁻ fluorescence spectrum 223 can be referred to as a spectral shift. Referring again to FIG. 1A, by monitoring the fluorescence spectrum 222 of the emission of photons 122, the radiative state of an NV center 102 can be monitored. As discussed below, a change in the parameter of interest can be detected based on a predetermined relationship between the parameter and the spectral shift. For example, the parameter of interest can be an electric field, a concentration of ions 112, or a pH level.

FIG. 1B is similar to FIG. 1A. The concentration of ions 112 can be greater in FIG. 1B than in FIG. 1A. A greater concentration of ions 112 near the surface 103 can result in a surface charge at the surface 103, which can induce an electromagnetic field across nanodiamond 101 and color center 102. As a result, the electron 111 shown in FIG. 1A can move from the electron trap (potential well) 113 shown in FIG. 1B) to the color center 102, which can cause the color center 102 to change to a negatively charged state in FIG. 1B. By way of example and not limitation, a pump field 121 can be applied to an NV center 102, which can be in a negatively charged state NV⁻. Referring also to FIG. 2, the resulting the emission of photons 123 can have a fluorescence spectrum 223, which can have a wavelength peaked over 637 nm, as further discussed below. The change in the concentration of ions 112 can be detected based on a predetermined relationship between the spectral shift and the ionic concentration, as further discussed below.

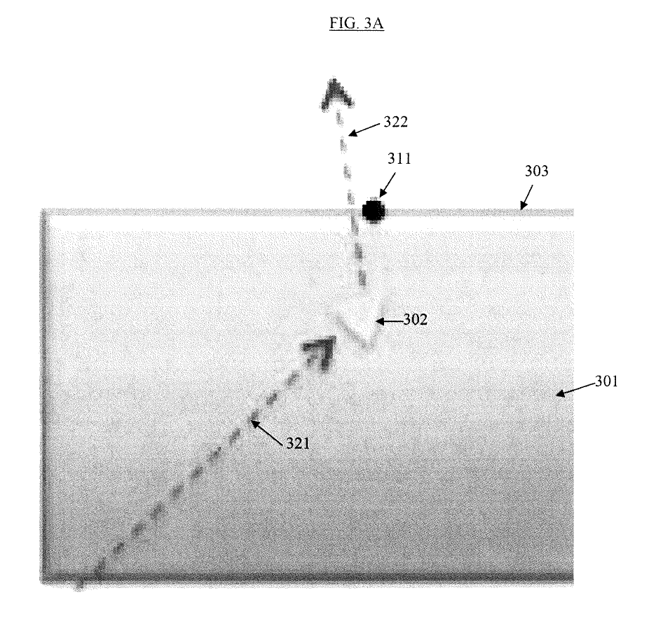

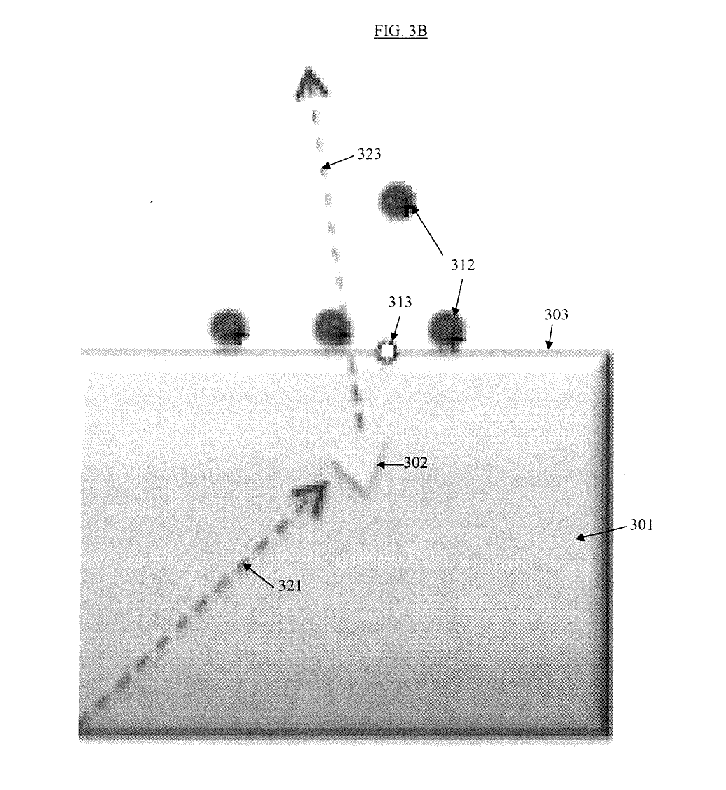

Referring to FIG. 3A, a bulk diamond crystal 301 can have a color center 302. The color center 302 could be any suitable color center, as discussed above. The color center 302 can be below the surface 303 of the bulk diamond crystal 301. The color center 302 can be any suitable depth below the surface 303, as discussed above.

By way of example and not limitation, the color center 302 can be an NV center. The charge state of NV center 302 can depend on the electrochemical potential seen by the electrons 311 on the diamond's surface 303 and surrounding environment.

An electromagnetic pump field 321 can be applied to color center 302, which in turn can cause color center 302 to emit photons 322. Referring also to FIG. 2, this emission of photons 322 can be detected to as the fluorescence spectrum 222. The fluorescence spectrum 222 can depend on the charge state of the color center 302, as discussed above. A monitoring device can be used to optically detect the fluorescence spectrum of NV center 302, as discussed above. A pump electromagnetic field can be applied to the NV center 302, as discussed above. By monitoring the fluorescence spectrum 222 of the emission of photons 322, the radiative state of an NV center 302 can be monitored. As discussed below, a change in the parameter of interest can be detected based on a relationship between the parameter and the spectral shift.

FIG. 3B is similar to FIG. 3A. The concentration of ions 312 can be greater in FIG. 3B than in FIG. 3A, which can induce an electromagnetic field across bulk diamond crystal 301 and color center 302. As a result, the electron 311 shown in FIG. 3A can move from the electron well 313 shown in FIG. 3B to the color center 302, which can cause the color center 302 to change to a negatively charged radiative state in FIG. 3B. By way of example and not limitation, a pump field 321 can be applied to an NV center 302, which can be in a negatively charged state NV⁻. Referring also to FIG. 2, the resulting the emission of photons 323 can have a fluorescence spectrum 223. The change in the concentration of ions 112 can be detected based on a predetermined relationship between the spectral shift and the ionic concentration, as further discussed below.

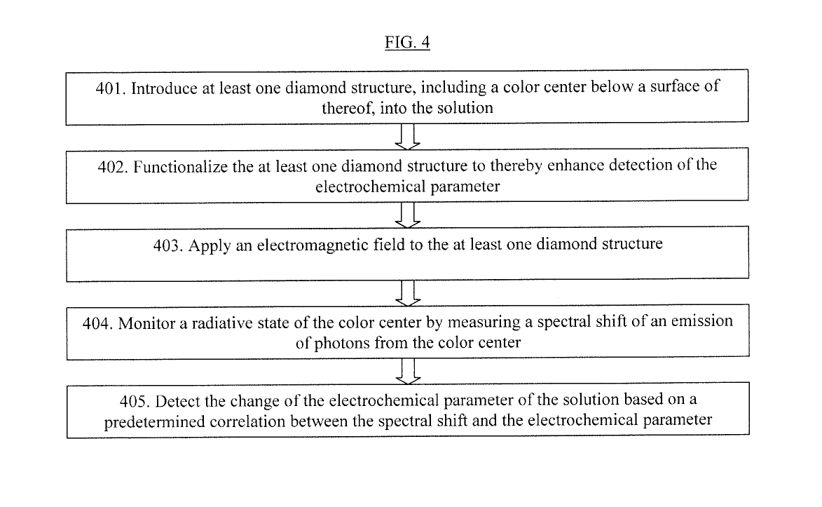

FIG. 4 is an exemplary diagram illustrating a method to detect a change of an electrochemical parameter in a solution, in accordance with some embodiments of the disclosed subject matter. At least one diamond structure can be introduced into a solution (401). For example, the diamond structure could be a nanodiamond 101 as in FIG. 1A-B or a bulk diamond crystal 301 as in FIG. 3A-B. The solution can be any type of solution. For example, the solution can be a biological solution, such as in a cell, a tissue, a vessel, or a lumen. For example, the solution could be any ionic solution, such as an electrolyte solution. Referring to FIG. 1A-B as an example for convenience, the diamond structure 101 can have a color center 102 below a surface 103 thereof. An electromagnetic pump field 121 can be applied to the color center 102 (403), as discussed below. A radiative state of the color center 102 can be monitored by measuring a spectral shift of an emission of photons 122 from the color center 102 (404). The change of a parameter of interest can be detected based on a correlation between the spectral shift and the parameter (405). For example, the parameter of interest can be an electric field, a concentration or ions 112, or a pH level. To enhance detection of the parameter, the diamond structure 101 can be functionalized (402).

By way of example and not limitation, the color center 102 can be an NV center. As discussed above, when an NV center 102 is near the surface 103 of the diamond structure 101, an electron 111 can move, or hop, from a trap state on the surface 103 to the NV center 102 or vice-versa. For example, electron 111 can be supplied from an electron donor, such as a nitrogen atom. As a result, the charge state of the NV center 102 can shift from NV⁰ to NV⁻ or vice versa. This hopping of the electron 111 and resulting shifting of the charge state of the NV center 102 can be referred to as blinking. This blinking can be optically detected by monitoring the emission of photons 122 and the corresponding fluorescence spectrum 222. In order to enhance detection of the parameter of interest, the diamond structure 101 can be functionalized (402) such that electron transfer occurs when the parameter crosses a threshold value based on a predetermined relationship between the parameter and the spectral shift. For example, the tendency of the electron 111 to populate the surface can depend on the conditions of the surface and its immediate surroundings.

As discussed further below, by modifying the surface charge density, the electron 111 can have a greater or lesser tendency to populate the surface state. For example, if the surface charge is made more positive, the electron 111 can have a greater tendency to populate the surface state, and more negative surface charge density can result in a lesser tendency. For example, the electron affinity of the diamond structure 101 can be modified by suitable preparation of the surface 103. A more electronegative surface 103 can strip electrons from the solution or the surrounding environment, which can result in a more negative surface charge on the surface 103 of the diamond structure 101 and a lesser tendency of the electron 111 to populate the surface state. Conversely, less electronegative can result in a more positive surface charge and a greater tendency of the electron 111 to populate the surface state. As discussed further below, by controlling the rate of transfer of charge from the NV center 102 to the surface 103, the spectral shift can occur when the parameter of interest crosses a threshold value based on a predetermined relationship between the parameter and the spectral shift.

By way of example and not limitation, the parameter of interest can be an electric field generated by a cell. For example, the parameter can be the electric field generated by a neuron. The color center 102 can be an NV center. The diamond structure 101 can be introduced into the cell or onto the surface of the cell (401). Before or after step 401, the diamond structure 101 can be functionalized to enhance detection of the electric field generated by the cell (402). For example, the electron affinity of the diamond structure 101 can be modified by preparing the surface 103 termination. A more electronegative surface 103 can strip electrons from the solution or the surrounding environment, which can result in a surface charge on the surface 103 of the diamond structure 101 and a corresponding electric field across the diamond structure 101 and the color center 102. Alternatively or additionally, an external electromagnetic field can be applied across the diamond structure 101. Alternatively or additionally, the pH of the solution can be modified by adding an acid or a base, and the change in pH can result in a change in the surface charge at the surface 103 of the diamond structure 101. The diamond structure 101 can be functionalized (402) such that the charge state of the NV center 102 is near the threshold where it will transition from NV⁰ to NV⁻ or vice-versa. As such, the electric field generated by the cell, e.g., the electric field change generated when a neuron fires, can combine with the electric field resulting from the functionalization, and the combined field can cause the NV center 102 to transition to a different charge state. For example, the NV center 102 can transition from NV⁰ to NV⁻. As discussed above, the fluorescence spectrum of the NV center 102 can shift when the NV center 102 changes charge states. An electromagnetic pump field 121 can be applied to the NV center 102 (403), as discussed below. A radiative state of the color center 102 can be monitored by measuring the spectral shift of the emission of photons 122 from the color center 102 (404). The change of the electric field generated by the cell can be detected based on a predetermined correlation between the spectral shift and the electric field generated by the cell (405).

By way of example and not limitation, the parameter of interest can be an ionic concentration in a solution. For example, the parameter can be the concentration of ions 112 in an electrolyte bath. The color center 102 can be an NV center. The diamond structure 101 can be introduced into the electrolyte bath (401). Before or after step 401, the diamond structure 101 can be functionalized to enhance detection of the concentration of ions 112 in the electrolyte bath (402). For example, the electron affinity of the diamond structure 101 can be modified by preparing the surface 103 to be in a different state of electronnegativity. A more electronegative surface 103 can strip electrons from the electrolyte bath, which can result in a surface charge on the surface 103 of the diamond structure 101 and a corresponding electric field across the diamond structure 101 and the color center 102. Alternatively or additionally, an external electromagnetic field can be applied across the diamond structure 101. Alternatively or additionally, the pH of the solution can be modified by adding an acid or a base, and the change in pH can result in a change in the surface charge at the surface 103 of the diamond structure 101. The diamond structure 101 can be functionalized (402) such that the charge state of the NV center 102 is near the threshold where it will transition from NV⁰ to NV⁻ or vice-versa. As discussed above, a change in concentration of ions 112 in the solution can result in an electric field across the diamond structure 101 and the NV center 102. As such, the electric field resulting from the concentration of ions 112 can combine with the electric field resulting from the functionalization, and the combined field can cause the NV center 102 to transition to a different charge state. For example, the NV center 102 can transition from NV⁰ to NV⁻. As discussed above, the fluorescence spectrum of the NV center 102 can shift when the NV center 102 changes charge states. An electromagnetic pump field 121 can be applied to the NV center 102 (403), as discussed below. A radiative state of the color center 102 can be monitored by measuring the spectral shift of the emission of photons 122 from the color center 102 (404). The change of the concentration of ions 112 in the electrolyte bath can be detected based on a predetermined correlation between the spectral shift and the concentration of ions 112 (405).

By way of example and not limitation, the parameter of interest can be a pH of a solution. The color center 102 can be an NV center. The diamond structure 101 can be introduced into the solution (401). Before or after step 401, the diamond structure 101 can be functionalized to enhance detection of the pH in the solution (402). For example, the electron affinity of the diamond structure 101 can be modified by preparing the surface 103. A more electronegative surface 103 can strip electrons from the electrolyte bath, which can result in a surface charge on the surface 103 of the diamond structure 101 and a corresponding electric field across the diamond structure 101 and the color center 102. Alternatively or additionally, an external electromagnetic field can be applied across the diamond structure 101. The diamond structure 101 can be functionalized (402) such that the charge state of the NV center 102 is near the threshold where it will transition from NV⁰ to NV⁻ or vice-versa. As discussed above, a change in the pH of the solution can result in a change in the surface charge at the surface 103 of the diamond structure 101 and a corresponding electric field across the diamond structure 101 and the NV center 102. As such, the electric field resulting from the pH of the solution can combine with the electric field resulting from the functionalization, and the combined field can cause the NV center 102 to transition to a different charge state. For example, the NV center 102 can transition from NV⁰ to NV⁻.

As discussed above, the fluorescence spectrum of the NV center 102 can shift when the NV center 102 changes charge states. An electromagnetic pump field 121 can be applied to the NV center 102 (403), as discussed below. A radiative state of the color center 102 can be monitored by measuring the spectral shift of the emission of photons 122 from the color center 102 (404). The change of the concentration of ions 112 in the electrolyte bath can be detected based on a predetermined correlation between the spectral shift and the concentration of ions 112 (405).

By way of example and not limitation, a plurality of diamond structures 101 can each be individually monitored (404), as discussed above. The change of the parameter of interest at each of the diamond structures 101 can be detected (405), as discussed above. Because the spectral shift can be a change in wavelength on the order of tens of nanometers, the charge state of each diamond structure that is in the NV⁰ state can be discriminated from the diamond structures in the NV⁻ state. As a result, relatively high fidelity detection of the parameter of interest can be achieved. For example, it is estimated that a change of 100V/cm can be detected after only 1 second of signal acquisition from a single NV center, and that a change of 10,000V/cm can be detected after 0.1 milliseconds of signal averaging. Using a larger number N of NV centers, the sensitivity can improve as 1/sqrt(N). In some embodiments, the diamond structures 101 can be nanodiamonds. The nanodiamonds 101 can be placed on a plurality of neurons. When an individual neuron fires, it can generate an electric field change. The electric field can be detected (405) by the nanodiamond 101 placed on that neuron in the manner discussed above. By monitoring each of the nanodiamonds 101 on each of the neurons (404) and detecting which of the neurons are generating an electric field at a given time (405), one can determine which neurons are firing at a given time. The robustness of this exemplary detection scheme can be enhanced with the use of large number of diamond structures 101 or using multiple NV centers 102 in each nanodiamond 101, for which known statistical methods can be applied to improve the signal to noise ratio by averaging over greater signal intensity.

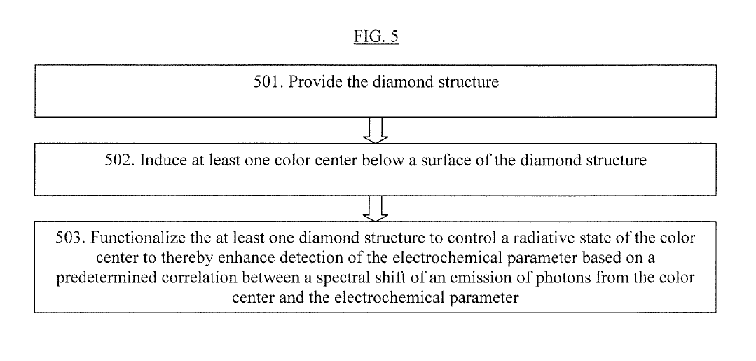

FIG. 5 is an exemplary diagram illustrating an exemplary method of fabricating a diamond structure for detecting a change of an electrochemical parameter at a surface thereof, in accordance with some embodiments of the disclosed subject matter. By way of example and not limitation, a diamond structure can be provided (501). The diamond structure can be formed or fabricated using known techniques. For example, the diamond structure can be fabricated by any of the techniques disclosed in commonly assigned U.S. Provisional Application No. 61/794,510, which is hereby incorporated by reference in its entirety. The diamond structure can have naturally occurring color centers therein. Alternatively or additionally, at least one color center can be deterministically induced below the surface of the diamond structure (502). For example, a color center can be induced by one of nano-implanting or electron radiating. The at least one diamond structure can be functionalized to control a radiative state of the color center to thereby enhance detection of the electrochemical parameter based on a predetermined correlation between a spectral shift of an emission of photons from the color center and the electrochemical parameter (503), as discussed above.

Referring again to FIG. 1A-B in connection with step 501, by way of example and not limitation, nanodiamonds 101 can be injected into a biological cell, a biological tissue, or the like. By way of example and not limitation, nanodiamonds 101 can be bonded to a tip of a micro-manipulated probe. By way of example and not limitation, nanodiamonds 101 can be bonded to a surface of a sample holder. By way of example and not limitation, nanodiamonds 101 can be bonded to a wall of a flow cell. For example, the flow cell can have an input, a flow channel, and an output, and the nanodiamonds 101 can be bonded to the walls of the flow channel. By way of example and not limitation, nanodiamonds 101 can be bonded to one of a tip of an optical fiber or a side of the optical fiber.

Referring again to FIG. 3A-B in connection with step 501, by way of example and not limitation, bulk diamond crystals 301 can be positioned below a sample. By way of example and not limitation, bulk diamond crystals 301 can be attached to a probe.

Referring again to FIG. 3A-B in connection with step 502, by way of example and not limitation, the diamond structure 301 can be a diamond wafer with a surface pattern (not pictured). A plurality of color centers 302 can be induced below the surface pattern of the diamond wafer 301. By way of example and not limitation, the color centers 302 can be in a geometric pattern. For example, the pattern of the color centers 302 can correspond to the surface pattern of the diamond wafer 301. Alternatively, the pattern of the color centers 302 can be unrelated to the surface pattern of the diamond wafer 301. By way of example and not limitation, the color centers 302 can be arranged arbitrarily.

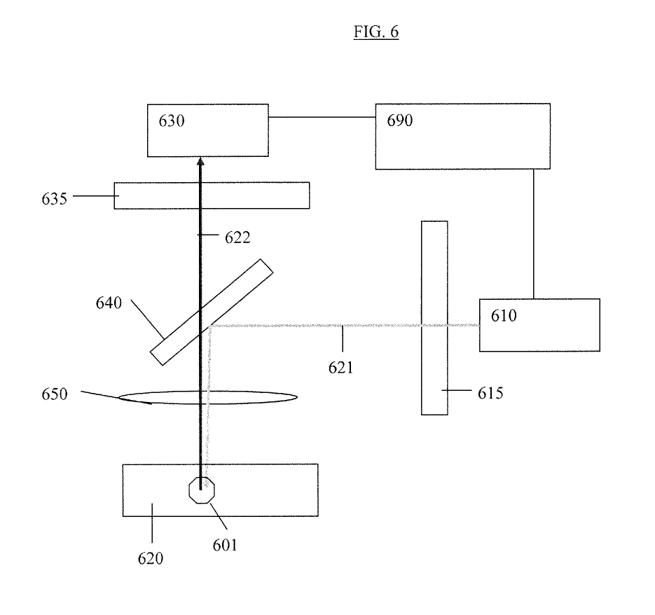

FIG. 6 is an exemplary diagram illustrating a system for detecting a change of an electrochemical parameter in a solution, in accordance with some embodiments of the disclosed subject matter. However, various modifications will become apparent to those skilled in the art from the following description and the accompanying figures. Such modifications are intended to fall within the scope of the appended claims.

By way of example and not limitation, a receptacle 620 can be adapted to receive the solution and at least one diamond structure 601. One or more of the diamond structures 601 can have a color center, for example as shown in FIGS. 1A-B and 3A-B. The diamond structure 601 can be at least partially submerged in a solution in receptacle 620. The solution can be a fluid, such as a biological solution, an ionic solution, or air. In some embodiments, the diamond structure 601 can be exposed to a solution within a biological cell, a biological tissue, or the like. For example, the receptacle can be a cell, a lumen or a tissue, and the diamond structure 601 can be introduced therein.

The diamond structure 601 can be optically pumped to excite the color centers contained therein. For example, the color centers can be NV centers. The diamond structure 601 can be continuously pumped with green laser at approximately 532 nm with a power near 1 mW focused to a 500 nm spot. For pulsed excitation, the power can scale down with the duty cycle. In some embodiments, optical pumping can occur at discrete times. For example, a pulse of pump light can be applied at each time that a readout is desired.

Optical pumping can be accomplished with a suitable electromagnetic pump field source 610, which can include a green laser capable of emitting light at 532 nm. Additional optics 615 and 635 can be employed to guide, filter, focus, reflect, refract, or otherwise manipulate the light. Such optics can include, for example, a pinhole aperture and/or barrier filter (not shown). Additionally, a dichromatic mirror 640 can be used to direct pump light to the receptacle 620 and diamond structure 601 while transmitting a fluorescent response. For example, the electromagnetic pump field source 610 can be a light source 610 and can be arranged such that pump light 621 is reflected off of a dichromatic mirror 640 and towards the receptacle 620 and diamond structure 601. A fluorescent response from the diamond structure 601 will be directed through the dichromatic mirror 640 in a direction orthogonal to the orientation of the light source 610.

The pump light 621 can be directed through an objective 650 to the diamond structure 601. Photons in the pump light 621 can be absorbed by the NV centers within the diamond structure 601 exposed to the receptacle 620, thereby exciting the NV center into an excited state, as discussed below. The NV can then transition back to the ground state, emitting fluorescent response 622, e.g., a photon with a wavelength between 637 and 600 nm. This fluorescent response can pass through the objective 650 and the dichromatic mirror 640 to a monitoring device 630. The monitoring device 630 can be a photodetector. In certain embodiments, the photodetector 630 can include a photomultiplier. The photodetector 630 can be, for example, an emCCD camera. Alternatively, the photodetector 630 can be a scanning confocal microscope or other suitable photon detector.

By way of example and not limitation, a plurality of diamond structures 601 can be distributed throughout the area of the receptacle 620. The area of the receptacle 620 can be divided into a number of pixels, each pixel corresponding to subset of the area. For each pixel, the fluorescent response 622 can be measured by the photodetector 630. In some embodiments, the control unit 890, which can include a processor and a memory, can calculate the parameter based on the fluorescent response 622 of the NV centers. In this manner, the parameter of interest can be detected at each pixel, as discussed above.

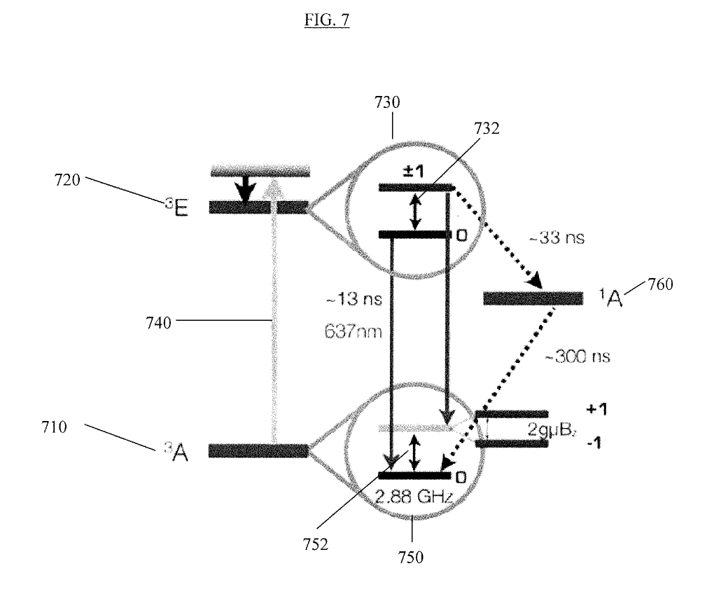

Referring to FIG. 7 an exemplary NV center is illustrated. NV centers can absorb photons 740 with a wavelength around 532 nm and emit a fluorescent (PL) response, which can be between 637 and 800 nm. A spin-dependent intersystem crossing 760 between excited state 720 triplet (3) to a metastable, dark singlet level 710 (S) can change the integrated PL for the spin states |0 [symbol] and |±1 [symbol] . The deshelving from the singlet 710 occurs primarily to the |0 [symbol] spin state, which can provide a means to polarize the NV center.

As depicted in FIG. 7, transitions from the NV ground state 710 to the excited state 720 are spin-conserving, keeping the magnetic sublevel quantum number, mₛ, constant. Such an excitation can be performed using laser light at approximately 532 nm 740; however, other wavelengths can be used, such as blue (480 nm) and yellow (580 nm). While the electronic excitation pathway preserves spin, the relaxation pathways contain non-conserving transitions involving an intersystem crossing (or singlet levels).

In accordance with the disclosed subject matter, the NV centers can be used to detect a parameter of interest, for example, for detecting electric fields, ionic concentrations, or pH, as discussed above. Detection of the parameter of interest can occur without detecting spin states in the diamond. Moreover, diamond nanoprobes with an NV center can be photostable. For example, single NV centers can emit without a change in brightness for months or longer. Additionally diamond is chemically inert, cell-compatible, and has surfaces that can be suitable for functionalization with ligands that target biological samples, as discussed above. NV centers can emit in excess of 106 photons per second, which can be relatively brighter than certain other light emitters.

The foregoing merely illustrates the principles of the disclosed subject matter. Various modifications and alterations to the described embodiments will be apparent to those skilled in the art in view of the teachings herein. It will thus be appreciated that those skilled in the art will be able to devise numerous techniques which, although not explicitly described herein, embody the principles of the disclosed subject matter and are thus within its spirit and scope.

1. A method to detect a change of an electrochemical parameter in a solution, comprising:
introducing at least one diamond structure, including a color center below a surface of thereof, into the solution;
applying an electromagnetic pump field to the at least one diamond structure;
functionalizing the at least one diamond structure to thereby enhance detection of the electrochemical parameter;
monitoring a radiative state of the color center by measurement of an emission of photons from the color center; and
detecting the change of the electrochemical parameter of the solution based on a predetermined correlation between the measurement and the electrochemical parameter.

2. The method of claim 1, wherein the functionalizing comprises modifying at least one of a surface charge density of the at least one diamond structure or an electron affinity of the at least one diamond structure.

3. The method of claim 1, wherein the diamond structure comprises one of a nanodiamond or a bulk diamond crystal.

4. The method of claim 1, wherein the solution comprises a biological solution or an ionic solution.

5. The method of claim 1, wherein the electrochemical parameter comprises one of an electric field, an ionic concentration, or a pH level.

6. The method of claim 1, wherein the measurement comprises measurement of a spectral shift of an emission of photons from the color center.

7. The method of claim 1, wherein the color center comprises a nitrogen vacancy (NV) center, the monitoring comprising monitoring a negatively charged radiative state of the NV center and a neutrally charged radiative state of the NV center.

8. The method of claim 1, wherein the color center comprises one of a silicon vacancy or a chromium center.

9. The method of claim 7, wherein the functionalizing comprises modifying at least one of a surface charge density of the at least one diamond structure or an ion affinity of the at least one diamond structure to control a charge transfer rate of the NV center to thereby enhance detection of the electrochemical parameter.

10. A system for detecting a change of an electrochemical parameter in a solution, comprising:
a receptacle adapted to receive the solution and at least one diamond structure having a color center below a surface of thereof, such that the diamond structure is at least partially submerged in the solution, the surface adapted to thereby enhance detection of the electrochemical parameter;
an electromagnetic pump field source adapted to apply an electromagnetic pump field to the at least one diamond structure; and
a monitoring device, coupled to the receptacle and adapted to monitor a radiative state of the color center by measurement of a spectral shift of an emission of photons from the color center to thereby detect the change of the electrochemical parameter of the solution based on a predetermined correlation between the measurement and the electrochemical parameter.

11. The system of claim 10, the surface adapted to enhance at least one of a surface charge density or an ion affinity to thereby enhance detection of the electrochemical parameter.

12. The system of claim 10, wherein the diamond structure comprises one of a nanodiamond or a bulk diamond crystal.

13. The system of claim 10, wherein the solution comprises a biological solution or an ionic solution.

14. The system of claim 10, wherein the electrochemical parameter comprises one of an electric field, an ionic concentration, or a pH level.

15. The system of claim 10, wherein the measurement comprises measurement of a spectral shift of an emission of photons from the color center.

16. The system of claim 10, wherein the color center comprises a nitrogen vacancy (NV) center, and wherein the monitoring device is adapted to monitor a negatively charged radiative state of the NV center and a neutrally charged radiative state of the NV center.

17. The system of claim 10, wherein the color center comprises one of a silicon vacancy or a chromium center.

18. The system of claim 5, the surface adapted to enhance at least one of a surface charge density or an ion affinity to control a blinking rate of the NV center to thereby enhance detection of the electrochemical parameter.

19. A method of fabricating a diamond structure for detecting a change of an electrochemical parameter at a surface thereof, comprising:
providing the diamond structure;
inducing at least one color center below a surface of the diamond structure; and
functionalizing the diamond structure to thereby enhance detection of the electrochemical parameter based on a predetermined correlation between a measurement of an emission of photons from the color center and the electrochemical parameter.

20. The method of claim 19, wherein the inducing comprises one of nano-implanting or electron radiating.

21. The method of claim 19, wherein the diamond structure comprises a nanodiamond, the providing comprising one of:
injecting the nanodiamond into one of a biological cell or a biological tissue;
bonding the nanodiamond to a tip of a micro-manipulated probe;
bonding the nanodiamond to a surface of a sample holder;
bonding the nanodiamond to a wall of a flow cell; or
bonding the nanodiamonds to one of a tip of an optical fiber or a side of the optical fiber.

22. The method of claim 19, wherein the diamond structure comprises a bulk diamond crystal, the providing comprising one of:
positioning the bulk diamond crystal below a sample; or
attaching the bulk diamond crystal to a probe.

23. The method of claim 19, wherein the diamond structure comprises a diamond wafer with a surface pattern, the inducing comprising inducing a plurality of color centers below the surface pattern of the diamond wafer.
